(19) United States
(12) Patent Application Publication
(10) Pub. No.: US 2025/0275722 A1
(43) Pub. Date: Sep. 4, 2025

(54) BIOSENSOR RELIABILITY JUDGMENT METHOD FOR WEARABLE DEVICES

(71) Applicant: Brandon Tyler Richey, Marion, IL (US)

(72) Inventor: Brandon Tyler Richey, Marion, IL (US)

(21) Appl. No.: 19/066,032

(22) Filed: Feb. 27, 2025

Related U.S. Application Data
(60) Provisional application No. 63/560,688, filed on Mar. 2, 2024.

(30) Foreign Application Priority Data
Sep. 26, 2024 (CN) 202411351953.2

(51) Int. Cl. A61B 5/00

(52) U.S. Cl. CPC A61B 5/6843; A61B 5/6824; A61B 5/6831; A61B 5/7264; A61B 5/746; A61B 2560/0462

(57) ABSTRACT

Wearable electronic devices can be equipped with sensors to measure heart rate, blood oxygen level, respiratory rate, blood pressure, blood sugar level, and skin temperature. The device can include graphene-based sensors to measure heart rate, blood oxygen level, respiratory rate, blood pressure, blood sugar level, and skin temperature. The device may include a process for judging the reliability of biological sensor measurements using a capacitance measurement. The capacitance measurement can be conducted on the device and can determine the quality of contact between the device and the wearer. The device can display warnings to the wearer when the quality of contact between the device and its wearer is not sufficient to conduct a reliable biological sensor measurement.

### CROSS-REFERENCE TO RELATED APPLICATION

This patent application claims the benefit of and priority to U.S. Provisional Patent Application No. 63/560,688, filed Mar. 2, 2024, and Chinese Patent Application No. 202411351953.2, filed Sep. 26, 2024, each of which is hereby incorporated by reference herein in its entirety.

### TECHNICAL FIELD

The present description relates to wearable devices and their biological sensor systems.

### BACKGROUND

Accurate biological data recording, processing, and presentation is key to understanding an individual's health. Wearable electronic devices can include sensors for biological data collection including blood pressure, skin temperature, blood sugar and other information. These biological sensors are typically configured and driven by software to give the wearer a user interface that reflects the state of their current health. The wearable biological sensors can be used along with other sensors, such as accelerometers, to determine the wearer's health in real-time during all types of activities. These biological sensor assemblies can be comprised of a variety of materials, including graphene.

### BRIEF DESCRIPTION OF THE DRAWINGS

Some features of the subject technology are described in the appended claims. In order to explain the subject technology, many embodiments are described in the following figures.

FIG. 1 illustrates a perspective view of a wearable electronic device, in accordance with evidence provided in the disclosure.

FIG. 2 illustrates a bottom-side view of a wearable electronic device, in accordance with evidence provided in the disclosure.

FIG. 3 illustrates a cross-sectional side view of a wearable electronic device, in accordance with evidence provided in the disclosure.

FIG. 4 illustrates an electronic circuit diagram of a biosensor capacitance measurement circuit inside a wearable electronic device, in accordance with evidence provided in the disclosure.

FIG. 5 illustrates a flow diagram of a biosensor measurement reliability process that uses wearable electronic devices, in accordance with evidence provided in the disclosure.

FIG. 6 illustrates a block diagram of a biosensor measurement system that uses wearable electronic devices, in accordance with evidence provided in the disclosure.

FIG. 7 illustrates a block diagram of an electronic device architecture that implements the aspects described in FIGS. 1-7.

### DETAILED DESCRIPTION OF THE EMBODIMENTS

The detailed description provided in this disclosure is to provide a description of the subject technology and is not representative of the only configuration of the subject technology. The drawings included in this disclosure serve as a part of the detailed description. The detailed description is intended to provide specific details regarding the subject technology. Skilled field professionals will understand that the details provided in this disclosure are not the only applicable uses and configurations for the subject technology. Block diagrams are utilized to describe components to clearly communicate the concepts of the subject technology.

Wearable electronic devices can include components such as a motherboard with a processing unit, random access memory, flash memory, biosensors, a battery, and other hardware which are parts of an assembly inside an enclosure. These electronic devices can also include user input devices such as a touch screen, which may also serve as a display for the electronic device. Application software can configure and control the electronic device's touch screen display to indicate the current date and time, provide biosensor measurement results, or execute other applications stored inside the memory of the electronic device.

A wearable electronic device can be attached to a user using a strap secured to the electronic device's enclosure. The electronic device's strap can be interchangeable with other straps of different sizes and/or colors. The contact between the user's skin and a wearable electronic device's biosensors is key to reliable biosensor measurements. If the wearable electronic device is not properly secured to the user, the information read from the electronic device's biosensors will become unreliable.

The subject technology described in this disclosure provides a biosensing method for wearable electronic devices utilizing graphene-based biosensors. The subject technology described in this disclosure may include graphene-based biosensor components that make direct contact with a user's skin to perform measurements. The subject technology described in this disclosure provides a method for performing biosensor measurements using graphene-based sensors inside a wearable electronic device.

Once a user decides to use a wearable electronic device for biosensing purposes, that user can launch an application that activates the components associated with the graphene biosensors. For example, a user can use the graphene biosensors to measure their blood pressure through wrist skin contact with the wearable electronic device. In another example, a user can use the graphene biosensors to measure their skin temperature through wrist skin contact with the wearable electronic device. In another example, a user can use the graphene biosensors measure their heart rhythm through wrist and finger skin contact with the wearable electronic device.

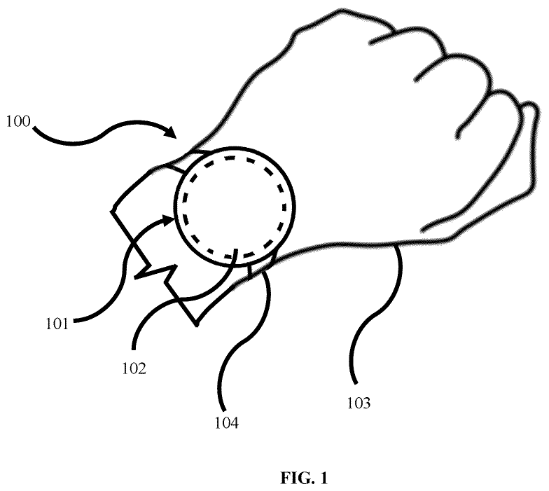

The subject technology is described in detail below with reference to FIGS. 1-6. Those who are skilled in the field will understand that the detailed description, along with its referenced figures, are meant for describing the subject technology, and not limiting the subject technology and its configurations.

FIG. 1 depicts a perspective view of a wearable electronic device that is attached to the body of a user. FIG. 1 shows a wearable electronic device 100 fastened to the wrist of a user 103 with a wristband 104 which can be made from fabric, metal, plastic, and/or other materials. The example in FIG. 1 depicts a wearable electronic device attached to the wrist of a user. However, configurations can take different forms such as a mobile device attached to a user using a band 104. For example, a mobile device can be a cellular phone, smart watch, medical device, location tracking device, and/or other electronic device.

FIG. 1 includes an enclosure 101 which contains the internal hardware of a wearable electronic device 100. The enclosure 101 depicted in the example of FIG. 1 is of a round shape, but the enclosure can be of other shapes. The internal hardware of a wearable electronic device can contain microprocessor, biosensor, location sensor, accelerometer, and cellular modem components and/or other electronic components. The wearable electronic device display 102 can include user touch input components which enable the user to interact with the wearable electronic device. The wearable electronic device display 102 may display date and time information for the user and/or other application information. The display 102 may be comprised of an assembly including a liquid crystal display, light emitting diode display, active-matrix organic light-emitting diode display and/or other display technologies. The display 102 may be attached to the top of the enclosure 101 and the wristband 104 may be attached to the enclosure 101 to form a wearable electronic device 100 fastened to the wrist of a user 103.

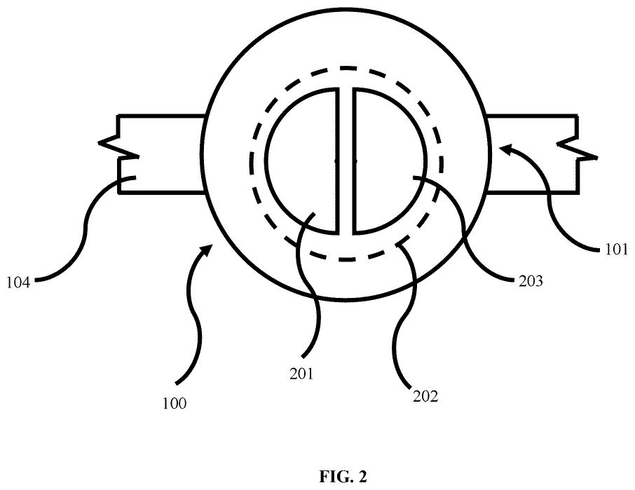

FIG. 2 depicts a perspective view of a wearable electronic device 100 to depict its biosensor hardware. FIG. 2 shows a wearable electronic device 100 with a positive polarity graphene biosensor wrist pad 201 and a negative polarity graphene biosensor wrist pad 203 mounted to its enclosure 101. A wearable electronic device 100 can be attached to a user with a wristband 104 to secure a user's wrist to a positive polarity graphene biosensor wrist pad 201, and a negative polarity graphene biosensor wrist pad 203 for biosensor measurements. The example in FIG. 2 depicts a positive polarity graphene biosensor wrist pad 201 and a negative polarity graphene biosensor wrist pad 203 connected to internal hardware 202 of a wearable electronic device 100. The internal hardware 202 of a wearable electronic device 100 may contain analog signal sampling, microprocessor, modem, location sensor, and haptic feedback driver components and/or other electronic components. The example in FIG. 2 shows the internal hardware 202 of a wearable electronic device 100 inside a wearable device enclosure 101. However, this example configuration is not limited and can be changed to feature certain hardware components mounted on the outside of an enclosure 101 of a wearable electronic device 100.

In certain examples, a positive polarity graphene sensor wrist pad 201 can be connected to a non-inverting input of an operational amplifier inside an analog signal sampling component. A negative polarity graphene sensor wrist pad 203 can be connected to an inverting input of an operational amplifier inside an analog signal sampling component. An analog signal sampling component can be used to sample analog signals for heart rate, blood oxygen, blood pressure, respiratory rate, blood sugar and skin temperature biosensor measurements and/or other biosensor measurements.

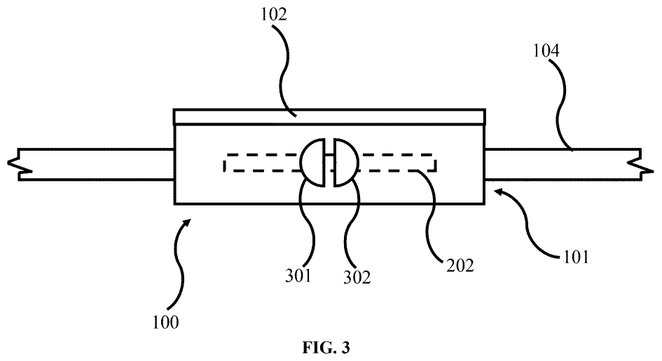

FIG. 3 depicts a cross-sectional side view of a wearable electronic device 100 and its internal electronic hardware 202 inside an enclosure of a wearable electronic device 101. The example in FIG. 3 shows the display 102 of a wearable electronic device 100 mounted on the top of a wearable electronic device enclosure 101. FIG. 3 shows a wearable electronic device 100 with a positive polarity graphene biosensor finger pad 301, and a negative polarity graphene biosensor finger pad 302 for biosensor measurements. The example in FIG. 3 depicts a positive polarity graphene biosensor finger pad 301 and a negative polarity graphene biosensor finger pad 302 connected to internal hardware 202 of a wearable electronic device 100. The example in FIG. 3 shows the internal hardware 202 of a wearable electronic device 100 inside a wearable device enclosure 101. However, this example configuration is not limited and can be changed to feature certain hardware components mounted on the outside of an enclosure 101 of a wearable electronic device 100.

In certain examples, a positive polarity graphene sensor finger pad 301 can be connected to a non-inverting input of an operational amplifier inside an analog signal sampling component. A negative polarity graphene sensor finger pad 302 can be connected to an inverting input of an operational amplifier inside an analog signal sampling component. A user can use a wearable electronic device 100 fastened to a user's wrist 103 with a wearable electronic device wristband 104 to perform biosensor measurements with a positive polarity graphene sensor wrist pad 201, negative polarity graphene sensor wrist pad 203, positive polarity graphene sensor finger pad 301, and a negative polarity graphene sensor finger pad 302 to perform biosensor measurements. A wearable electronic device 100 with a positive polarity graphene sensor wrist pad 201, negative polarity graphene sensor wrist pad 203, positive polarity graphene sensor finger pad 301, and a negative polarity graphene sensor finger pad 302, can be used to perform heart rate, blood oxygen, blood pressure, respiratory rate, blood sugar and skin temperature biosensor measurements and/or other biosensor measurements.

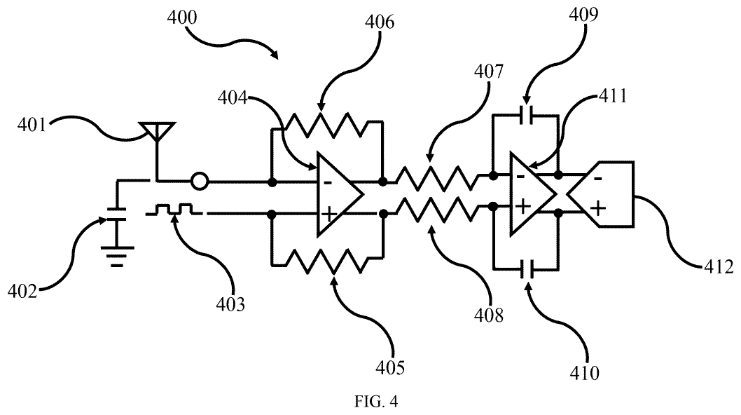

FIG. 4 depicts an electronic circuit diagram of a biosensor capacitance measurement circuit 400 inside a wearable electronic device 100. The biosensor capacitance measurement circuit comprises a graphene biosensor pad 401 connected to a user 103. A graphene biosensor pad can be connected to an inverting input of a transimpedance operational amplifier 404. A transimpedance operational amplifier 404 gain can be controlled using a feedback resistor 406 connected between the inverting input and inverting output of a transimpedance operational amplifier 404, and a feedback resistor 405 connected between the non-inverting input and non-inverting output of a transimpedance operational amplifier 404. An inverting output of a transimpedance operational amplifier 404 can be connected to an integrator operational amplifier 411 input resistor 407, and a non-inverting output of a transimpedance operational amplifier 404 can be connected to an integrator operational amplifier 411 input resistor 408. An inverting integrator operational amplifier 411 feedback capacitor 409 can be connected between an inverting input of an integrator operational amplifier 411, and an inverting output of an integrator operational amplifier 411, and a non-inverting integrator operational amplifier 411 feedback capacitor 410 can be connected between a non-inverting input of an integrator operational amplifier 411, and a non-inverting output of an integrator operational amplifier 411. An inverting output of an integrator operational amplifier 411 can be connected to an inverting input of an ADC (Analog to Digital Converter) component 412, and a non-inverting output of an integrator operational amplifier 411 can be connected to a non-inverting input of an ADC component 412. The example in FIG. 4 is not limited, and can include operational amplifier components, analog to digital components, and/or other electronic components.

In certain examples, a transimpedance operational amplifier's gain can be controlled using feedback resistors 405 and 406. The relationships between the inputs and outputs of a transimpedance operational amplifier 403 can be determined as V_{OUT}=−i_{IN}R_{F} for an inverting input, and V_{OUT}=i_{IN}R_{F} for a non-inverting input. A reference pulse signal 403 can be applied to a non-inverting input of a transimpedance operational amplifier 403, which controls the sequencing for a biosensor sampling circuit. The feedback capacitors 409 and 410 can be connected to an integrator operational amplifier 411 for electrical current integration. The relationships between the inputs and outputs of an integrator operational amplifier 411 can be determined as

$${V}_{OUT} = - \frac{1}{{R}_{INT} {C}_{INT}} {\int }_{0}^{t} {v}_{IN} ( t ) dt$$

for an inverting input, and

$${V}_{OUT} = \frac{1}{{R}_{INT} {C}_{INT}} {\int }_{0}^{t} {v}_{IN} ( t ) dt$$

for a non-inverting input. The outputs of integrator operational amplifier 411 are connected to the ADC component 412 for sequential analog voltage sampling and digital conversion. As the user's contact with the biosensor fluctuates, the user's body capacitance 402 measured by the ADC component 412 will vary as well. The example in FIG. 4 is not limited and can include operational amplifier input and output relationships and/or other electrical circuit relationships.

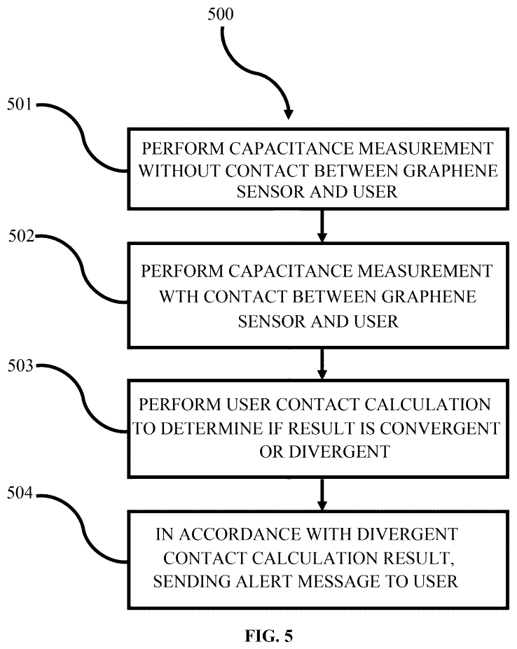

FIG. 5 depicts a flow diagram of a biosensor measurement reliability process using a wearable electronic device 100. The example in FIG. 5 shows a method that can be used to determine the reliability of a graphene biosensor measurement using a wearable electronic device 100. A biosensor measurement reliability algorithm can be used to determine the quality of contact between a user and a biosensor mounted on a wearable electronic device 100. The graphene biosensor measurement reliability method 500 depicted in example FIG. 5 is not restrictive and can be applied to electronic devices such as cellular phones, tablet computers, smart watches, fitness trackers, medical devices and/or other electronic devices.

In certain examples, a series of capacitance measurements without user skin contact 501 are taken to calculate an average value and used as a baseline for the graphene biosensor measurement reliability method 500. A capacitance measurement can be taken with user skin contact 502, and a difference, or delta, between a measured value with biosensor user skin contact and a reference capacitance value without biosensor user skin contact can be calculated. The delta between the reference capacitance value without user skin contact and the measured capacitance value with user skin contact can be used as an input for a graphene biosensor user contact reliability calculation 503. A set of Lorenz characteristic equations can be used to calculate the quality of contact between a user and a graphene biosensor mounted on a wearable electronic device 100. An example application of the Lorenz characterisuc equations

$$\frac{dx}{dt} = σ ( - x + y ) , \frac{dy}{dt} = rx - y - xz , and \frac{dz}{dt} = - bz + xy$$

can be used to determine if a capacitance biosensor measurement is convergent or divergent. A delta between a capacitance reference value without user contact with a wearable electronic device 100 graphene biosensor, and a measured capacitance value with user contact with a wearable electronic device 100 graphene biosensor, can be used as an input variable r in a set of Lorenz characteristic equations. A set of critical point values in the form of (x,y,z) can be chosen, a value for variable o can be chosen, and a value for variable b can be chosen to solve a set of Lorenz characteristic equations. In an example, a critical point of (3,8,0), σ=10,

$$b = \frac{8}{3} ,$$

and a Jacobian matrix J(x,y,z)=

$$( \begin{matrix}Fx & Fy & Fz \\ Gx & Gy & Gz \\ Hx & Hy & Hz\end{matrix} ) = ( \begin{matrix}- σ & σ & 0 \\ r - z & - 1 & - x \\ y & x & - b\end{matrix} ) where \frac{dx}{dt} = F ( x , y , z ) , \\ \frac{dy}{dt} = G ( x , y , z ) , and \frac{dz}{dt} = H ( x , y , z )$$

can yield a characteristic polynomial

$$- {λ}^{3} - \frac{41}{3} {λ}^{2} - \frac{145}{3} λ + 10 λ r + \frac{80}{3} r - \frac{1070}{3} .$$

An eigenvalue of the characteristic polynomial

$$- {λ}^{3} - \frac{41}{3} {λ}^{2} - \frac{145}{3} λ + 10 λ r + \frac{80}{3} r - \frac{1070}{3}$$

can be determined as

$$λ = \frac{1}{3} ( - \frac{{2}^{\frac{1}{3}} ( - 270 r - 376 )}{3 {( 9 \sqrt{15} \sqrt{- 64800 {r}^{3} - 114660 {r}^{2} + 5002636 r + 46186191} - 13770 r - 237337 )}^{\frac{1}{3}}} + \frac{1}{3 \sqrt[3]{2}} ( {( 9 \sqrt{15} \sqrt{- 64800 {r}^{3} - 114660 {r}^{2} + 5002636 r + 46186191} - 13770 r - 237337 )}^{\frac{1}{3}} ) - \frac{41}{3} ) .$$

In the example of FIG. 5, the variable r can be set to −63 for proper user contact with a graphene biosensor on a wearable electronic device 100, which can yield an eigenvalue of λ=−3.15686, and the variable r can be set to −38 for improper user contact with a graphene biosensor on a wearable electronic device 100, which can yield an eigenvalue of λ=−3.48748. An eigenvalue for improper user contact with a graphene biosensor on a wearable electronic device 100, λ=−3.15686, can be included in a judgment threshold to determine the quality of contact between a user and a positive polarity graphene biosensor wrist pad 201, a negative polarity graphene biosensor wrist pad 203, a positive polarity graphene sensor finger pad 301, and/or a negative polarity graphene sensor finger pad 302 connected to internal hardware 202 of a wearable electronic device 100. Eigenvalues that are within a judgment threshold are considered convergent, and eigenvalues that are outside of a judgment threshold are considered divergent. A convergent capacitance biosensor calculation can be used to determine that the contact between a user and a graphene biosensor mounted on a wearable electronic device 100 is sufficient for a reliable biosensor measurement. A divergent capacitance biosensor measurement can be used to determine that the contact between a user and a graphene biosensor mounted on a wearable electronic device 100 is insufficient for a reliable biosensor measurement. The example method shown in FIG. 5 is not limited to one specific calculation method, and other calculation methods can be used. In the event of insufficient contact between a user and a graphene biosensor mounted on a wearable electronic device 100, an alert can be issued to the user wearing the wearable electronic device 504 for notification. A user biosensor contact alert can be issued using a display 102 and/or speakers mounted on a wearable electronic device 100.

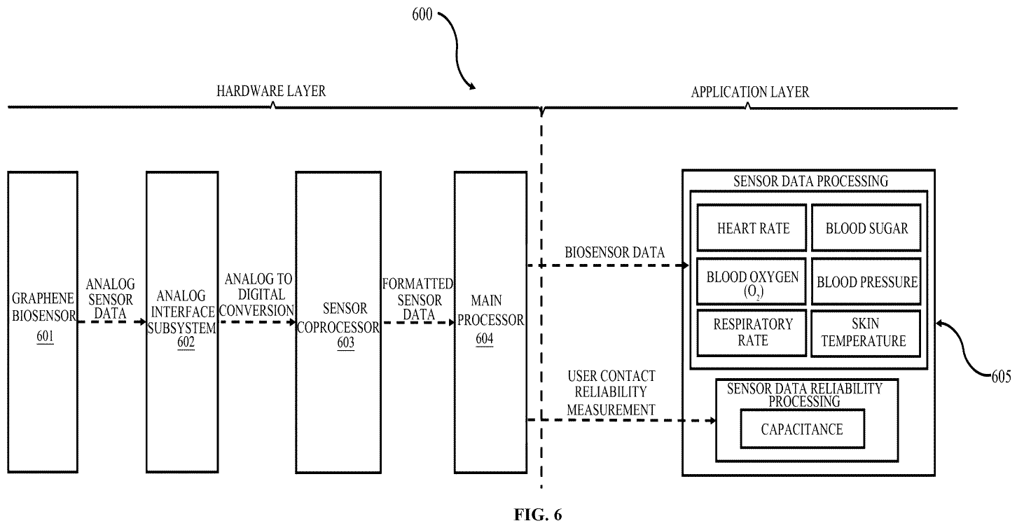

FIG. 6 depicts a block diagram of a biosensor measurement system 600 for a wearable electronic device 100. The example in FIG. 6 shows a wearable electronic device 100 biosensor measurement system 600 hardware layer and application layer. The example in FIG. 6 is not restrictive and a biosensor measurement system 600 can be comprised of hardware layers, application layers and/or other layers.

In certain examples, a graphene biosensor 601 mounted on a wearable electronic device 100 can be connected to a user to read analog data. An analog interface subsystem 602 can be used to read analog sensor data from a graphene biosensor 601. An analog interface subsystem 602 of a wearable electronic device 100 can include Analog Front End (AFE) integrated circuitry with internal operational amplifiers. Operational amplifier settings within an AFE integrated circuit can be configurable with software programming. Analog Front End integrated circuitry can include programmable filters with the purpose of signal noise reduction. However, digital filters with the purpose of signal noise reduction are only an example since digital filters can be used for signal reconstruction and/or other signal processing applications. An analog interface subsystem 602 can include ADC circuitry for analog signal sampling and digital value conversion. The precision of ADC sampling circuitry can be configured with software programming. Digital data from a sampled analog signal can be sent to a sensor coprocessor 603. A sensor coprocessor can be used to format and organize sensor data sent from an analog interface subsystem 602. The example in FIG. 6 is not restrictive and a sensor coprocessor can be used for sensor data organization, sensor data formatting and/or other data processes. A main processor 604 can be used to receive formatted sensor data from a sensor coprocessor 603. A main processor 604 can perform high-level computations to present the sensor data to a user wearing a wearable electronic device 100. The example in FIG. 6 is not restrictive and a main processor 604 can be used for high-level data computations for presentation and/or other computational functions.

The example in FIG. 6 depicts an application layer that interfaces with the hardware layer of a biosensor measurement system 600 for a wearable electronic device 100. In certain examples, heart rate, blood sugar, blood oxygen, blood pressure, respiratory rate, and skin temperature biosensor data can be presented to a user with a sensor data processing component 605 inside a wearable electronic device 100. The sensor data processing component 605 is comprised of application instructions that, when executed on a wearable electronic device 100, perform operations pertaining to heart rate, blood sugar, blood oxygen, blood pressure, respiratory rate, and skin temperature biosensor measurements and/or other biosensor measurements. A biosensor measurement reliability algorithm 503 can be executed using application software running on a wearable electronic device 100. A user biosensor contact reliability calculation 503 could produce negative results, which can prompt a message to be displayed on a wearable electronic device display 102 to inform the user of improper skin contact with a wearable electronic device's 100 biosensor(s). A user biosensor contact reliability calculation 403 could produce positive results, which can allow a biosensor measurement to be taken and displayed on a wearable electronic device display 102.

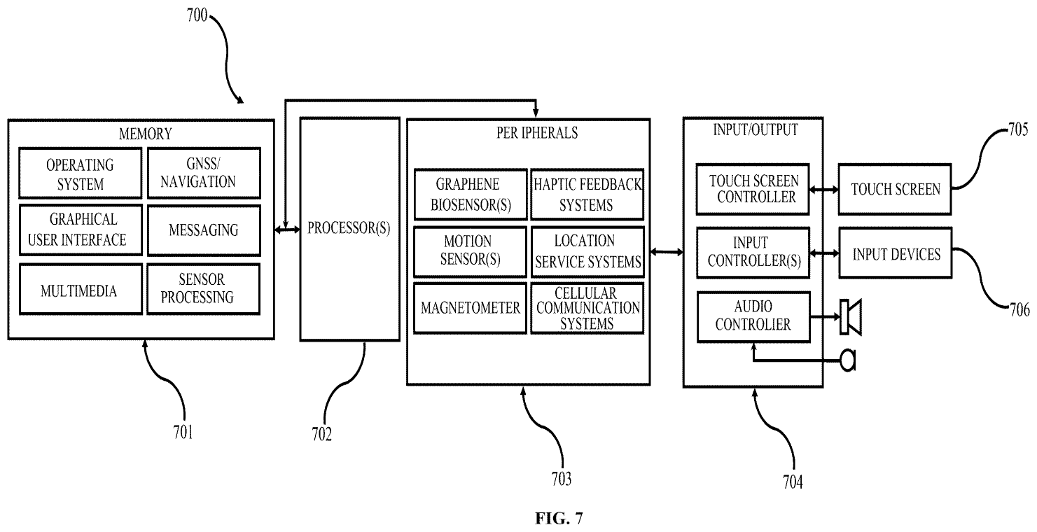

FIG. 7 depicts a block diagram of an electronic device architecture 700 that includes components that can be found on a wearable electronic device 100. The example in FIG. 7 shows an electronic device architecture 700 comprising memory 701, processor 702, peripheral 703, input/output 704, touch screen 705, and input device 706 components which can be used to implement a graphene biosensor system for a wearable electronic device 100. The example in FIG. 7 is not restrictive, and an electronic device architecture 700 can include memory 701, processor 702, peripheral 703, input/output 704, touch screen 705, and input device 706 components and/or other components.

In certain examples, a memory 701 component of an electronic device architecture 700 can be used to store and execute instructions for operating system, navigation, graphical user interface, messaging, multimedia, sensor processing applications and/or other applications. A memory 701 component can store sensor processing application instructions to perform biosensor measurements using graphene biosensor(s). A wearable electronic device 100 can display biosensor information to a user with an operating system using a graphical user interface running from a memory 701 component. A wearable electronic device 100 can execute multimedia instructions running from a memory 701 component to communicate biosensor result data to a user with graphics and sound. Messaging application instructions running from a memory 701 component can be executed to send alerts to a wearable electronic device 100 or other electronic device from one user to another, which can be biosensor alerts and/or other alerts. GNSS and/or other navigation application instructions can be executed from a memory 701 module to display the geographical location to a user and/or relay the geographical location of one user to another. The example in FIG. 7 is not restrictive and a memory 701 module can be comprised of a read-only memory (ROM) and/or random-access memory (RAM).

A processor 702 component can be used to perform computations for operating system, GNSS/navigation, graphical user interface, multimedia, messaging, and sensor processing applications found in a memory 701 component of a wearable electronic device 100. A processor 702 component can be used to perform computations for graphene biosensor, motion sensor, magnetometer, haptic feedback, location service, and cellular communications peripherals 703 found inside a wearable electronic device 100. The example in FIG. 7 is not restrictive and a processor 702 component can be comprised of a main processor and/or other processors.

Peripherals 703 of an electronic device architecture 700 can be comprised of graphene biosensor, motion sensor, magnetometer, haptic feedback, location service, and cellular communications peripherals and/or other peripherals. Graphene biosensor peripheral(s) can be used to perform user heart rate, blood oxygen, blood pressure, respiratory rate, blood sugar and skin temperature biosensor measurements and/or other biosensor measurements. Motion sensor peripheral(s) can be used to detect acceleration and/or deceleration of a wearable electronic device 100. Magnetometer peripheral(s) can be used to detect magnetic fields that are exposed to a wearable electronic device 100. Haptic feedback peripheral(s) can be used to activate and/or deactivate feedback motors inside a wearable electronic device 100. Location service peripheral(s) can be used to determine the geographical location of a wearable electronic device 100. Cellular communication peripheral(s) can be used to enable a wearable electronic device 100 to connect to cellular networks. The example in FIG. 7 is not restrictive and peripherals 703 can be comprised of graphene biosensor, motion sensor, magnetometer, haptic feedback, location service, and cellular communications peripherals and/or other peripherals.

Input/output (I/O) 704 components of a wearable electronic device 100 can be comprised of touch screen controller(s), input controller(s), audio controller(s) and/or other I/O devices. A touch screen controller can be used to enable a user to interact with a wearable electronic device display 102 with the use of a connected touch screen 705. An input controller can be used to enable a user to connect pushbutton switches, dials and/or other input devices 706 mounted on a wearable electronic device 100. An audio controller can be used to connect speakers, microphones and/or other audio devices. The example in FIG. 7 is not restrictive and I/O 704 components can be comprised of touch screen controller(s), input controller(s), audio controller(s) and/or other I/O devices.

The title, background, brief description of the drawings, abstract, and drawings included in this disclosure are to provide examples and illustrations of the subject technology and are not restrictive descriptions. The examples in this disclosure that contain combined elements are not to be restricted to the single combination of elements presented. The claims are incorporated into the detailed description for each individual subject matter separately. The claims are not limited to the described subject matter but are to adhere to a scope consistent with all legal equivalent language. The claims in this disclosure are not intended to include subject matter that does not meet the requirements of applicable patent law and should not be interpreted in this manner.

What is claimed is:

1. A wearable electronic device, comprising:
an enclosure with an internal cavity;
a mounted graphene biosensor component on the bottom side of the enclosure; and
a sensor processing component that is configured to obtain user health data.

2. The wearable electronic device of claim 1, wherein the graphene biosensor component comprises a first biosensor lead and a second biosensor lead.

3. The wearable electronic device of claim 1, wherein the graphene biosensor leads make contact with a user's wrist to obtain user health data.

4. The wearable electronic device of claim 1, wherein the graphene biosensor leads are secured to a user with a wrist strap.

5. A method comprising:
performing a capacitance measurement without user contact with a wearable electronic device biosensor; and
performing a capacitance measurement with user contact with a wearable electronic device biosensor; and performing a calculation to determine if the user contact is reliable or unreliable; and in accordance with the user contact being unreliable, sending an alert to one or more devices.

6. The method of claim 5, wherein the user contact reliability calculation further comprises: a delta capacitance calculation used as an input to a Lorenz polynomial; computing an eigenvalue to determine convergence or divergence; in accordance with the Lorenz polynomial being divergent; sending an alert to one or more devices; in accordance with the Lorenz polynomial being convergent, recording a biosensor measurement.

7. A system comprising:
at least one processor;
at least one graphene biosensor;
a wireless communication component;
a memory component containing instructions that when executed by the at least one processor perform operations comprising: reading user health data using the at least one graphene biosensor;
determining if a user has proper contact with the at least one graphene biosensor;
in accordance with the user not having proper contact with the at least one graphene biosensor, sending an alert to the user; and
in accordance with the user having proper contact with the at least one graphene biosensor, storing the biosensor measurement data in the memory component.

8. The system of claim 7, wherein the memory component further comprises: instructions that when executed by the at least one processor perform: reading user health data using the at least one graphene biosensor; determining if a user has proper contact with the at least one graphene biosensor; in accordance with the user not having proper contact with the at least one graphene biosensor, transmitting an alert to an electronic device using the wireless communication component.

9. The system of claim 7, wherein the memory component further comprises: instructions that when executed by the at least one processor perform: reading user health data using the at least one graphene biosensor; determining if the user health data is within a specified range; in accordance with the health data not being within a specified range, storing the biosensor measurement data in the memory component, sending an alert to the user; in accordance with the health data being within a specified range, storing the biosensor measurement data in the memory component.

10. The system of claim 7, wherein the memory component further comprises: instructions that when executed by the at least one processor perform: reading user health data using the at least one graphene biosensor; determining if the user health data is within a specified range; in accordance with the health data not being within a specified range, storing the biosensor measurement data in the memory component, transmitting an alert to an electronic device using the wireless communication component.
